(19) United States
(12) Patent Application Publication
(10) Pub. No.: US 2024/0415395 A1
(43) Pub. Date: Dec. 19, 2024

(54) EAR-WORN EDDY CURRENT MEASUREMENT DEVICE AND EAR-WORN SENSOR

(71) Applicant: NATIONAL TSING HUA UNIVERSITY, Hsinchu City (TW)

(72) Inventor: Ting-Wei WANG, Hsinchu City (TW)

(73) Assignee: NATIONAL TSING HUA UNIVERSITY, Hsinchu City (TW)

(21) Appl. No.: 18/582,310

(22) Filed: Feb. 20, 2024

(30) Foreign Application Priority Data
Jun. 16, 2023 (TW) 112122704

(51) Int. Cl. A61B 5/0205; A61B 5/00; A61B 5/024; A61B 5/145

(52) U.S. Cl. CPC A61B 5/0205; A61B 5/14532; A61B 5/6816; A61B 5/02438

(57) ABSTRACT

An ear-worn eddy current measurement device includes a sensor and a control module. The sensor includes a coil and a setting structure combined with the coil. The setting structure is configured to arrange the coil on an ear of a subject and place an emission surface of the coil towards an ear measurement area of the subject. The control module is coupled to the coil, and is configured to drive the coil to perform eddy current induction measurement on the measurement area and obtain at least one physiological information of the subject.

### TECHNICAL FIELD

The present invention relates to an ear-worn eddy current measurement device and an ear-worn sensor. Specifically, the present invention relates to an car-worn eddy current measurement device and an car-worn sensor for measuring physiological information.

### BACKGROUND

With the aging of the population and changes in dietary habits, the proportion of people suffering from chronic diseases such as diabetes or cardiovascular diseases is also gradually increasing. In order to relieve the burden on the medical system, it is necessary to develop home care or remote care. Therefore, the measurement mechanism of important parameters for diabetes or cardiovascular diseases, such as blood glucose levels or physiological information such as heart rate and blood pressure, will affect the success of the development of home care or remote care.

For the physiological information of cardiovascular diseases, wearable cardiovascular sensing devices have become the mainstream of current clinical medicine research because of their advantages such as continuous and long-term monitoring. There are already a variety of wearable sensors in current products that can be used to monitor cardiovascular parameters including continuous blood pressure or heart rate. However, although many teams have proposed and practiced the research and product development of wearable cardiovascular sensors, these sensors still have some limitations in use. For example, existing wrist-worn cardiovascular sensors all use photoplethysmography (PPG) mechanisms to measure heart rate or blood pressure. However, PPG technology still has several technical issues. For example, the low penetration of light into the skin can only extract physiological information about capillaries or superficial arterial pulses. In addition, different skin colors also cause differences in the depth of light penetration into the skin. The most important thing is that if the strap holding the sensor slips, a gap will be created between the PPG sensor and the skin, making it impossible for the PPG sensor's sensing light to penetrate into the blood vessel area to measure heart rate or blood pressure, and affect the continuity and accuracy of monitoring. In another example, the existing chest-worn cardiovascular sensing device still needs to be fixed on the subject with a strap, which may cause inconvenience and discomfort to the subject and affect the willingness of the subject to wear it for long-term observation.

For the measurement of blood glucose, currently common commercially available blood glucose machines usually use an invasive needle stick method to extract the blood and then perform blood glucose concentration analysis, which causes inconvenience to the patient and the pain of bleeding from the needle stick. Alternatively, microneedles are used to insert into the subcutaneous tissue and absorb skin tissue fluid, to detect whether blood glucose exceeds the standard. Although this method of using microneedles can slightly reduce the pain of blood extraction compared to the traditional needle stick method, it is still an invasive measurement technology.

### SUMMARY

One objective of the present invention is to provide a physiological information measurement device that is easy to wear while not easily influenced by the wearer's actions.

Another objective of the present invention is to provide a measurement device that is non-intrusive and capable of long-term monitoring of blood glucose value.

Yet another objective of the present invention is to provide a measurement device that is capable of detecting blood glucose information and cardiovascular information at the same time.

The present invention provides an ear-worn eddy current measurement device including a sensor and a control module. The sensor includes a coil and a setting structure combined with the coil. The setting structure is configured to arrange the coil on an car of a subject and place an emission surface of the coil towards an car measurement area of the subject. The control module is coupled to coil, and is configured to drive the coil to perform eddy current induction measurement on the car measurement area and obtain at least one physiological information of the subject.

The present invention also provides an car-worn sensor including a coil and a setting structure combined with the coil. The setting structure is configured to arrange the coil on an car of a subject and place an emission surface of the coil towards an car measurement area of the subject.

As described above, by disposing the sensor on the subject's car, the measurement influence caused by the subject's action can be reduced. Moreover, disposal on the car is less likely to affect the subject's action or living than disposal on the limbs or chest. Using a coil to perform eddy current induction measurement on the car (for example, car blood vessels or subcutaneous tissue fluid), the subject's physiological signals can be obtained. Compared with optical mechanisms, the eddy current measurement mechanism is not susceptible to physical limitations of skin color or barriers. Compared with blood sampling to measure blood glucose, the eddy current measurement mechanism is non-invasive and will not cause discomfort to the subject. Simultaneous eddy current induction measurement of car blood vessels or subcutaneous tissue fluid can also obtain blood glucose information and cardiovascular information at the same time, that is favorable to the development of home care or remote care.

### BRIEF DESCRIPTION OF THE DRAWINGS

The drawings presented in the present invention are intended to assist in describing various embodiments of the present invention. However, in order to simplify the drawings and/or highlight the content to be presented in the drawings, conventional structures and/or elements in the drawings may be drawn in a simple schematic manner or may be omitted. On the other hand, the number of elements in the drawings may be singular or plural. The drawings presented in the present invention are for the purpose of illustrating the embodiments only and are not limiting thereof.

FIG. 1 is a schematic diagram of an ear-worn eddy current measurement device in the first embodiment of the present invention.

FIGS. 2A to 2D are schematic diagrams of setting structures of an ear-worn sensor in the first embodiment of the present invention.

FIG. 3 is a schematic diagram of a coil performing eddy current induction measurement in the first embodiment of the present invention.

FIG. 4 is an exemplary block diagram of a control module in the second embodiment of the present invention.

FIG. 5 is an exemplary block diagram of a control module in the third embodiment of the present invention.

FIG. 6 is a schematic diagram of a direct current part and an alternating current part of a sensing signal in the third embodiment of the present invention.

FIG. 7 is a schematic diagram of direct current part calculation of a sensing signal in the third embodiment of the present invention.

FIG. 8 is an exemplary block diagram of a control module in the fourth embodiment of the present invention.

### DETAILED DESCRIPTION

Any reference herein to elements using names such as “first”, “second”, etc. generally does not limit the number or order of these elements. Rather, these names are used herein as a convenient way to distinguish between two or more elements or instances of elements. Therefore, it should be understood that the names “first”, “second”, etc. in the claims do not necessarily correspond to the same names in the written description. Furthermore, it should be understood that reference to first and second elements does not imply that only two elements may be employed or that the first element must precede the second element. The words “comprise”, “include”, “have”, “contain”, etc. used herein are all open terms, which mean including but not limited to.

The term “couple” used herein refers to direct or indirect electrical coupling between two structures. For instance, in an example of indirect electrical coupling, one structure may be coupled to another structure via passive devices like resistors, capacitors or inductors.

In the present invention, the words “exemplary” and “for example” are used to mean “used as an example, instance or illustration”. Any implementation or aspect described herein as “exemplary” or “for example” is not necessarily to be construed as “preferred or advantageous over other aspects of the invention”. The terms “approximately” and “roughly” as used herein with respect to a specified value or characteristic are intended to mean within a certain numerical value (e.g., 10%) of the specified value or characteristic.

### First Embodiment

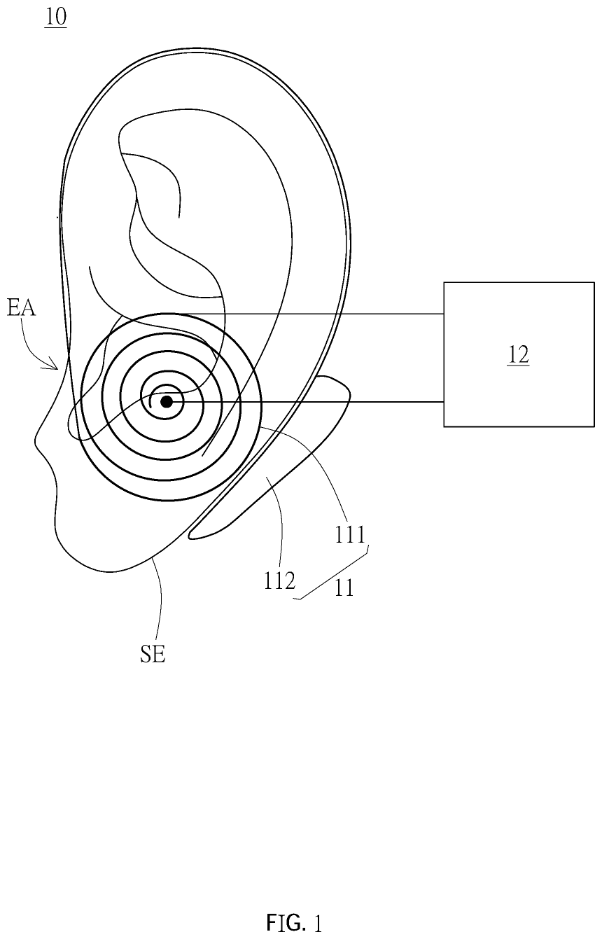

Refer to FIG. 1, an ear-worn eddy current measurement device 10 including a sensor 11 and a control module 12 is illustrated. The sensor 11 includes a coil 111 and a setting structure 112 combined with the coil 111. The setting structure 112 is configured to arrange the coil 111 on an ear SE of a subject and place an emission surface of the coil 111 towards an ear measurement area EA of the subject. The control module 12 is coupled to the coil 111, and is configured to drive the coil 111 to perform eddy current induction measurement on the ear measurement area EA and obtain at least one physiological information of the subject.

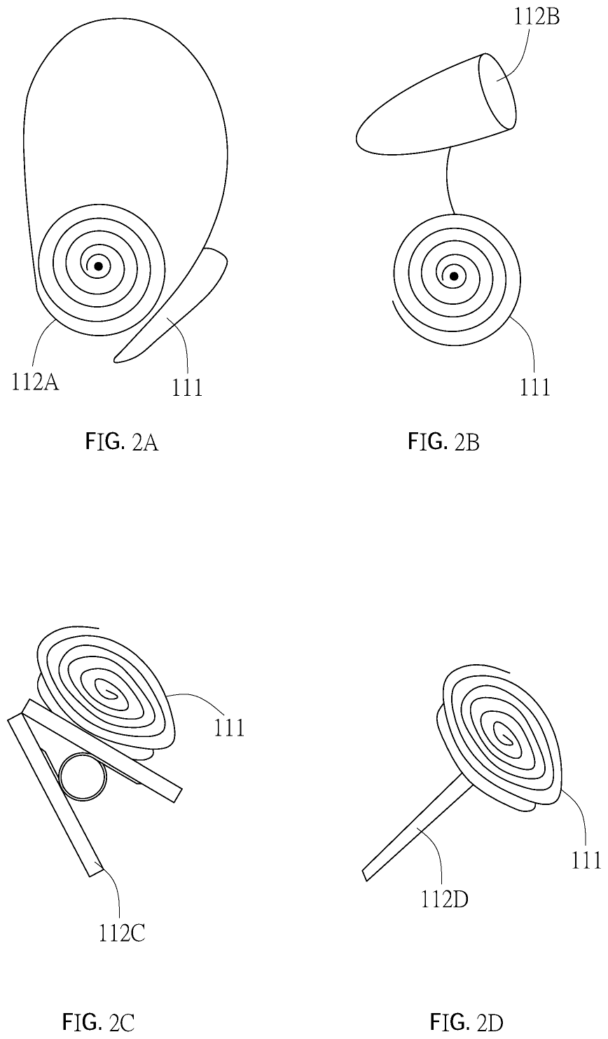

The coil 111 may be arranged on the ear SE of the subject by the setting structure 112. For example, the setting structure 112 may be of hook type, bud type, clip type or piercing type. Specifically, as shown in FIG. 2A, a hook type setting structure 112 has a hook structure 112A that may hang or hook on an outer ear part (e.g., helix and/or auricle) of the subject, and the coil 111 is arranged on the ear SE through a supporting force provided to the setting structure 112 by the outer ear part. As shown in FIG. 2B, a bud type setting structure 112 has a bud structure 112B like an earphone or earbud, the bud structure 112B is capable of arranging the coil 111 on the ear SE through a supporting force provided to the setting structure 112 by being plugged into the ear canal entrance and/or leaned against by the tragus of the subject. As shown in FIG. 2C, a clip type setting structure 112 may have a clip structure 112C that may clip locations like an outer ear part (e.g., earlobe and/or auricle) to provide a supporting force for arranging the setting structure 112. As shown in FIG. 2D, a piercing type setting structure 112 may make use of a pierced hole on an outer ear part of the subject, to provide to the setting structure 112 a supporting force needed for arranging the coil 111 by a stud structure 112D passing through the pierced hole. It should be noted that the exemplary setting structures 112 in FIGS. 2A to 2D is for the purpose of illustrating the setting structure 112 and not for limiting the present invention. Any means capable of arranging the coil 111 on the ear SE of the subject and placing an emission surface of the coil 111 towards the ear measurement area EA of the subject, such as adhesion, adsorption or tying, should all belong to the scope of the setting structure 112 of the present invention.

The coil 111 may be a conductor wiring formed on a substrate. Specifically, known fabrication techniques like etching, engraving, photolithography may be used to form the conductor wiring on the substrate. The formed conductor wiring has at least a radiating portion to output electromagnetic signals and receive feedback electromagnetic signals from the ear measurement area EA. The coil 111 may be of coil pattern like single-turn coil, multi-turn coil or helical coil, but is not limited thereto. Furthermore, the coil 111 on the substrate may be planar, for example, coil pattern formed with conductor wiring on a layer of the substrate. In another aspect, the coil 111 on the substrate may be stereoscopic, for example, coil pattern formed with conductor wiring in multiple layers of the substrate. Fabricating the coil 111 by known or conventional circuit fabrication means may effectively enhance fabrication yield and uniformity of the coil 111, and facilitate integration with other circuit devices and modules. However, the coil 111 may be a standalone component without the need to be disposed on a substrate. For example, the coil 111 may be wound with enameled wire (by example only and not limiting the material thereof). The coil 111 may be made of different materials, different numbers of turns, different shapes and other types of coils with different radiating portions according to the purpose.

An emission surface of the coil 111 is placed towards the ear measurement area EA of the subject. Specifically, the normal direction of the emission surface of the coil 111 is the direction with the highest directivity or the most intense energy among electromagnetic signals transmitted by the coil 111. The emission surface of the coil 111 may be obtained according to electromagnetic field assessment means like simulation or magnetic line. The ear measurement area EA is a position located on the ear SE of the subject and having blood vessels, capillaries or tissue fluid. In a preferred example, the ear measurement area EA is an earlobe, so that a smoother measurement surface may be obtained, and it may be less likely for measurements of the coil 111 to be influenced by variations in ear cartilage or helix structure due to simple structure of the earlobe position. In a preferred example, the ear measurement area EA is the back of the auricle, so that the coil 111 disposed on the back of the auricle is less likely influenced by the subject's action, and the privacy or aesthetics of the subject can be further protected. In another aspect, the emission surface of the coil 111 may have a matching layer or a blocking layer. The magnetic impedance of the matching layer lies between those of the ear measurement area EA and the coil 111, so that the loss caused by large difference in impedance during electromagnetic signal propagation can be reduced. The blocking layer may be a material capable of blocking electromagnetic waves such as conductor sheet, and may have a notch corresponding to the location on the emission surface with the highest directivity or the most intense energy among electromagnetic signals transmitted by the coil 111, so that directivity of the coil 111 can be enhanced. Furthermore, materials like buffer layer may also be disposed between the emission surface of the coil 111 and the ear measurement area EA, so that comfort of wearing can be increased, and the coil 111 is less likely displaced due to the subject's action.

The control module 12 is coupled to the coil 111. For example, the control module 12 may be coupled to the coil 111 on a substrate. Specifically, the control module 12 and the coil 111 may be disposed on the same substrate or different substrates. For example, the coil 111 may be formed on the same substrate and connected to the control module 12 disposed on the substrate with conductor wiring(s) on the substrate. Necessary active/passive devices of the control module 12 may be disposed on the substrate, e.g., by means of soldering. In one aspect, the standalone control module 12 is coupled to the car-worn sensor 11 by a transmission line, and may be worn on any part of the subject's body. In another aspect, when space of the ear-worn sensor 11 (e.g., housing for integration or space of the setting structure 112) allows, the control module 12 may also be integrated with the setting structure 112 or housing of the car-worn sensor 11, leading to a more integral car-worn eddy current measurement device 10 with improved convenience for the subject. However, the present invention is not limited to the connection manner between the control module 12 and the car-worn sensor 11; the connection manner should take the size, weight or wearing convenience and comfort of the control module 12 into consideration.

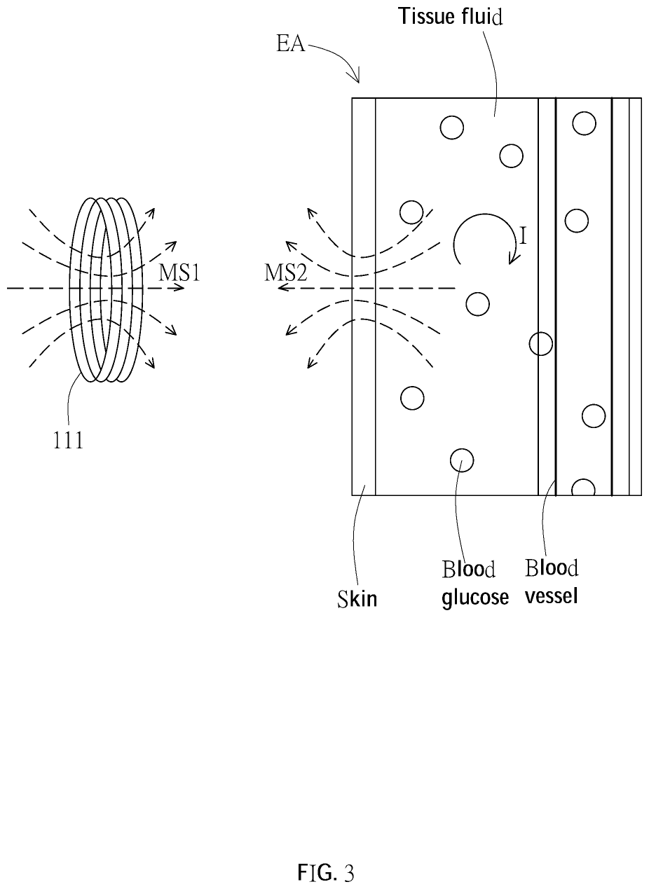

The control module 12 drives the coil 111 to perform eddy current induction measurement on the ear measurement area EA. Specifically, referring to FIG. 3, the control module 12 causes the coil 111 to generate a first electromagnetic signal MSI due to electromagnetic effect, and the first electromagnetic signal MS1 is transmitted to the ear measurement area EA. Because the tissue fluid or blood in the blood vessel within the ear measurement area EA has ions and is conductive, the tissue fluid or blood in the blood vessel can be regarded as a planar conductor. When the tissue fluid or blood in the blood vessel receives the first electromagnetic signal MS1, it will couple out induced electric field and generate a corresponding eddy current I. The magnitude of the eddy current I can vary depending on the conductivity or the cross-sectional area or volume of the planar conductor. Eddy currents I of different magnitudes generate a different second electromagnetic signal MS2 corresponding to the first electromagnetic signal MSI and in the opposite direction due to electromagnetic effect. By measuring the difference between the first electromagnetic signal MSI and the second electromagnetic signal MS2, the current state of the tissue fluid or blood in the blood vessel can be calculated. It should be noted that the difference between the first electromagnetic signal MS1 and the second electromagnetic signal MS2 can be obtained, for example but not limited to, through the variation in the inductance value of the coil 111 itself or by directly measuring the variation in the second electromagnetic signal MS2 (e.g., frequency, amplitude variation).

The current state of tissue fluid or blood in the blood vessel can correspond to at least one physiological information of the subject. In terms of blood vessel, because the blood in the blood vessel is driven by the contraction and relaxation of the heart, the amount of blood in the blood vessel will vary with the heart rate, which will cause the magnitude of the eddy current I induced by the first electromagnetic signal MS1 to vary with the heart rate. Therefore, the subject's heart rate can be estimated from the current state of blood in the blood vessel. However, the physiological information related to blood/blood vessel of the subject is not limited to the heart rate. “Blood/blood vessel-related physiological information” in the present invention is, for example but not limited to, vasoconstriction and/or diastole, pulse, blood vessel elasticity, intravascular status (for example, whether the inside of the blood vessel is blocked or unobstructed, blood flow status, blood flow velocity, etc.), blood vessel proliferation, blood vessel density, blood vessel wall status (for example, whether the blood vessel wall is damaged) and other medical/non-medical parameters. In another aspect, in terms of tissue fluid, the conductivity of the tissue fluid may be correlated with the subject's physiological information (e.g., blood glucose level). In other words, the subject's physiological information (for example, high or low blood sugar level) will affect the conductivity of the tissue fluid. For example, under the measurement condition of 10 MHz frequency, the conductivity difference between the blood/tissue fluid of normal people and diabetic patients is about 40%. When the tissue fluid generates the eddy current I in response to the first electromagnetic signal MS1, the conductivity of the tissue fluid will affect the magnitude of the eddy current I. Therefore, a corresponding model can be established between conductivity and blood glucose. For example, through a prosthesis or phantom with tissue fluid or blood, after measuring different blood glucose values, a correspondence relationship of the difference between the first electromagnetic signal MSI and the second electromagnetic signal MS2 and the physiological parameters can be obtained, thereby providing a correction or correspondence table to establish a measurement correspondence model. However, the physiological information related to tissue fluid of the subject is not limited to heart rate. “Physiological information related to tissue fluid” in the present invention includes, but is not limited to, blood glucose, inflammation status, osmotic pressure or other physiological information related to tissue fluid.

By disposing the sensor 11 on the subject's ear, the measurement influence caused by the subject's action can be reduced. Compared with being placed on the limbs or chest, the sensor 11 disposed on the ear is less likely to affect the subject's movement or life. By using the coil 111 to perform eddy current induction measurement on the ear (for example, ear blood vessels or subcutaneous tissue fluid), the subject's physiological information SI can be obtained without physical limitations such as skin color or barriers. Moreover, the eddy current measurement mechanism is non-invasive and will not cause discomfort to the subject. Simultaneous eddy current induction measurement of ear blood vessels or subcutaneous tissue fluid can also obtain blood glucose information and cardiovascular information at the same time. This allows the subject to obtain measurement results without wearing multiple devices, which can greatly increase the subject's willingness to wear it and contribute to the development of home care or long-term care.

### Second Embodiment

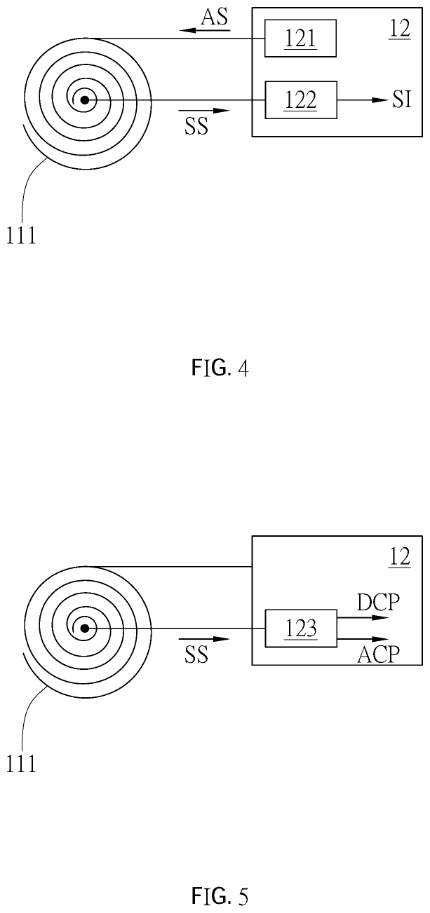

In this embodiment, as shown in FIG. 4, the control module 12 may include a signal generating unit 121 and a processing unit 122 coupled to the coil 111. The signal generation unit 121 may be an alternating current/direct current signal generation unit composed of active devices (e.g., oscillator, timer) and/or passive devices (e.g., resistor, capacitor, inductor). For example, the signal generation unit 121 may directly generate an alternating current signal AS through active/passive devices. In another aspect, the signal generation unit 121 may convert the direct current signal into the alternating current signal AS through a circuit of active/passive devices. In the example where the signal generation unit 121 converts the direct current signal into the alternating current signal, the signal generation unit 121 may include a direct current supply source and a resonant circuit. The resonant circuit receives the direct current signal provided by the direct current supply source to generate the alternating current signal AS. Through generating the alternating current signal AS by a direct current signal source and a resonant circuit, because the resonant circuit only requires a series/parallel combination of passive devices (e.g., resistors, capacitors, inductors), effects like simplified circuit and power saving can be achieved. The frequency of the alternating current signal AS of the present invention, or the frequency at which the coil 111 transmits the first electromagnetic signal MS1, is about 1-10 MHZ, so that the physiological information SI can have a better response.

After the signal generation unit 121 provides the alternating current signal AS to the coil 111, the coil 111 generates the first electromagnetic signal MSI due to electromagnetic effect. The coil 111 outputs the first electromagnetic signal MSI to the car measurement area EA so that the car measurement area EA generates the eddy current I. The eddy current I will generate the second electromagnetic signal MS2 with a magnetic field direction opposite to that of the first electromagnetic signal MS1. The second electromagnetic signal MS2 will be received by the coil 111. In other words, the second electromagnetic signal MS2 (alone or after interacting with the first electromagnetic signal MSI and/or other signals) generates a magnetoelectric effect on the coil 111 to generate the sensing signal SS.

The processing unit 122 of the control module 12 may perform, for example, sample or analog to digital conversion on the sensing signal SS, and then perform computation or measurement through devices with computing capabilities. The processing unit 122 may perform signal analysis on the sensing signal SS to obtain at least one physiological information SI of the subject corresponding to the sensing signal SS.

By using individual signal generation units and processing units to respectively excite the coil 111 and analyze signals of the coil 111, mutual interference during excitation and reception can be reduced. The coil 111 can also be switched between transmitting and receiving through a switch or other means to reduce the heating effect of the coil 111 due to the excited current or reduce the mutual interference during excitation and reception, thereby improving signal resolution and signal-to-noise ratio.

### Third Embodiment

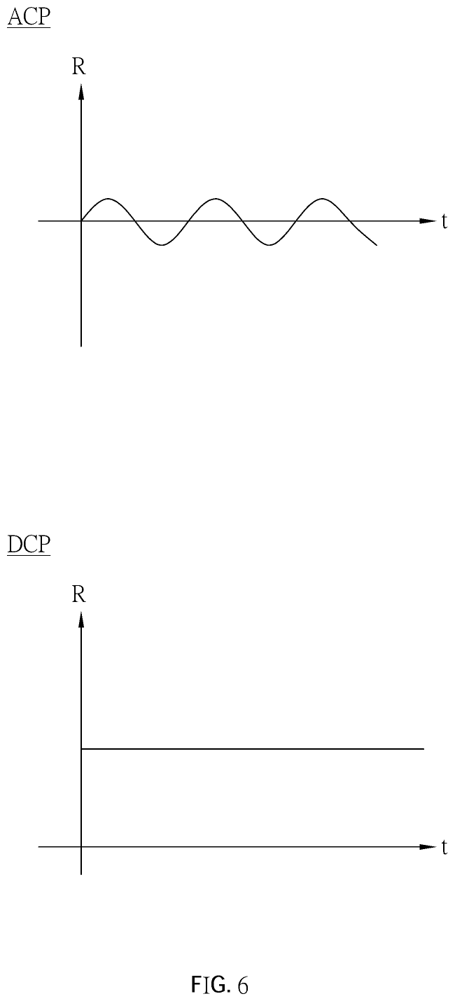

In this embodiment, as shown in FIG. 5 and FIG. 6, the control module 12 may include a filter unit 123 coupled to the coil 111. The filter unit 123 is configured to partition the sensing signal SS provided by the coil 111 into an alternating current part ACP and a direct current part DCP. For example, the filter unit 123 that receives the sensing signal SS may have two output channels corresponding to the output alternating current part ACP and the output direct current part DCP. The alternating current output channel can remove the direct current part DCP in the sensing signal SS through means such as inductors or filters (e.g., high pass, band pass). In another aspect, the direct current output channel can remove or reshape the alternating current part ACP of the corresponding signal into direct current through means of capacitors, low-pass filters or rectifiers. It should be noted that the X-axis in FIG. 6 represents time, the number of measurements, or time-dependent parameters. The Y-axis represents a response R, which is a measurable parameter such as voltage value, current value, inductance change, frequency change, etc. Moreover, the value, position, and waveform of the response R are only for illustration and are not intended to limit the response R of the signal of the present invention.

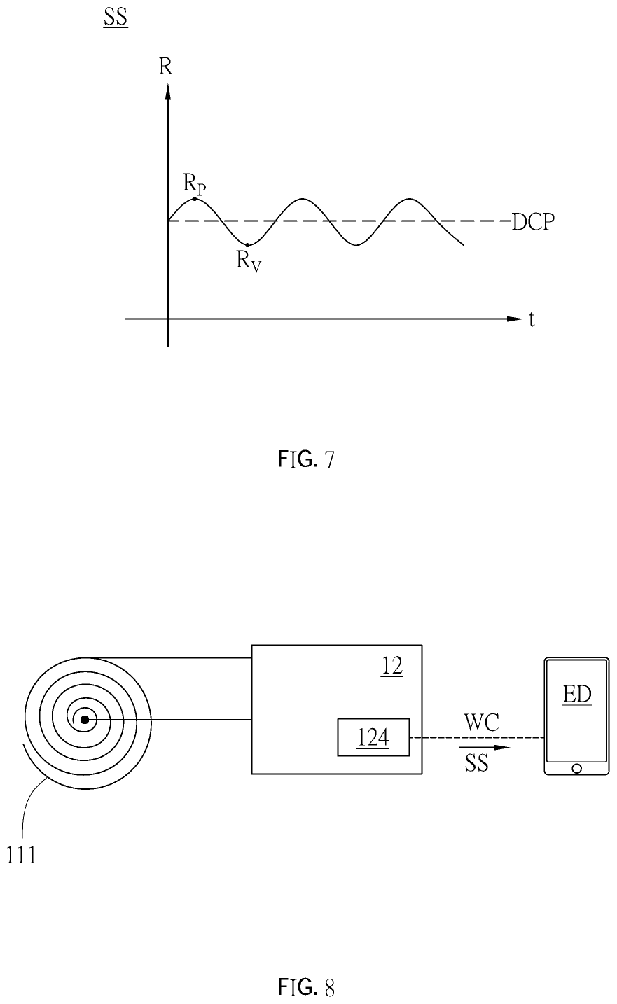

As described in the first embodiment, the alternating current part ACP may correspond to the frequency-containing part of the subject's physiological information SI, such as heart rate or pulse. The direct current part DCP may correspond to signal differences caused by different conductivities in the subject's physiological information SI, such as subcutaneous tissue fluid information like blood glucose. However, in the present invention, the computation of the direct current part DCP can also be obtained by directly computing the sensing signal SS. For example, refer to FIG. 7 and the following Formula (1):

$$DCP = \frac{\sum {R}_{pn} - \sum {R}_{vn}}{2} . \qquad Formula ( 1 )$$

In Formula (1), the peak values Rp and trough values Rv of the sensing signal SS are added together and subtracted from each other to calculate an intermediate value to obtain the direct current part DCP. It should be noted that the mutual subtraction of the peak values and the trough values can be combined with conventional operations such as averaging, integration or square averaging to obtain the direct current part DCP from the sensing signal SS.

The direct current part DCP and the alternating current part ACP of the sensing signal SS can be separated in advance by the filter unit 123. Subsequent signal processing, signal amplification or signal analysis can be adjusted according to different frequencies and different types of parts. Therefore, the filter unit 123 can be used as a good signal pre-processing means to facilitate subsequent signal processing.

### Fourth Embodiment

In this embodiment, as shown in FIG. 8, the control module 12 may include a communication unit 124. The communication unit 124 is configured to provide the physiological information SI obtained by measurement to the electronic device ED through a wireless terminal WC. Specifically, the electronic device ED is, for example, a back-end device such as a smartphone, a desktop computer, or a notebook computer. The communication unit 124 communicates with the electronic device ED through wireless means (e.g., Bluetooth, wireless network, infrared, etc.) and provides the at least one physiological information SI to the electronic device ED. An application program can be installed in the electronic device ED to record or analyze the at least one physiological information SI. This can be used for tracking, remote care or home care purposes.

It should be noted that all the embodiments of the present invention are not mutually exclusive. Embodiments may be combined with each other or implemented separately. For example, the control module 12 may include technical features of the second embodiment to the fourth embodiment at the same time. That is, the sensing signal SS is first pre-processed by the filter unit 123, computed by the processing unit, and then provided to the electronic device by the communication unit 124.

The preceding description of the present invention is provided so that a person of ordinary skill in the art can produce or implement the present invention. For a person of ordinary skill in the art, various modifications to the present invention will be obvious, and without departing from the spirit or scope of the present invention, the general principles defined herein can be applied to other variants. Therefore, the present invention is not intended to be limited to the examples described herein, but should be accorded the broadest scope of the principles and novel features disclosed herein.

what is claimed is:

1. An ear-worn eddy current measurement device, comprising:
a sensor including:
a coil; and
a setting structure combined with the coil, the setting structure configured to arrange the coil on an ear of a subject and place an emission surface of the coil towards an ear measurement area of the subject; and
a control module coupled to the coil, the control module configured to drive the coil to perform eddy current induction measurement on the ear measurement area and obtain at least one physiological information of the subject.

2. The ear-worn eddy current measurement device of claim 1, wherein the ear measurement area is located on an ear lobe of the subject.

3. The ear-worn eddy current measurement device of claim 1, wherein the eddy current induction measurement includes:
transmitting, with the coil, a first electromagnetic signal to the ear measurement area; and
receiving, by the coil, a feedback electromagnetic signal from the ear measurement area to obtain a sensing signal.

4. The ear-worn eddy current measurement device of claim 1, wherein the control module includes:
a filter unit coupled to the coil, the filter unit configured to partition a sensing signal provided by the coil into an alternating current part and a direct current part.

5. The ear-worn eddy current measurement device of claim 4, wherein the at least one physiological information includes a heart rate signal of the subject, and the heart rate signal corresponds to the alternating current part.

6. The ear-worn eddy current measurement device of claim 4, wherein the at least one physiological information includes subcutaneous tissue fluid information of the subject, and the subcutaneous tissue fluid information at least corresponds to the direct current part.

7. The ear-worn eddy current measurement device of claim 4, wherein the at least one physiological information includes subcutaneous tissue fluid information of the subject, and the subcutaneous tissue fluid information corresponds to an intermediate value calculated from peak values and trough values of the alternating current part.

8. The ear-worn eddy current measurement device of claim 1, wherein the control module includes:
a signal generation unit coupled to the coil, the signal generation unit configured to generate an alternating current signal, and provide the alternating current signal to the coil for generating a first electromagnetic signal; and
a processing unit coupled to the coil, the processing unit configured to receive a sensing signal from the coil, and calculate the at least one physiological information according to the sensing signal.

9. The ear-worn eddy current measurement device of claim 1, wherein the at least one physiological information includes heart rate or blood glucose of the subject.

10. The ear-worn eddy current measurement device of claim 1, wherein the control module includes:
a communication unit configured to output the at least one physiological information to an electronic device.

11. An ear-worn sensor, comprising:
a coil; and
a setting structure combined with the coil, the setting structure configured to arrange the coil on an ear of the subject and place an emission surface of the coil towards an ear measurement area of the subject.

12. The ear-worn sensor of claim 11, wherein the ear measurement area is located on an ear lobe of the subject.

13. The ear-worn sensor of claim 11, wherein the coil is configured to perform eddy current induction measurement on the ear measurement area and obtain at least one physiological information of the subject.
